(19) United States
(12) Patent Application Publication
(10) Pub. No.: US 2010/0065445 A1
(43) Pub. Date: Mar. 18, 2010

(54) Pre-Packaged, Customized, Woven, Wet Towel

(75) Inventor: Hugo Stevenson, Northridge, CA (US)

Correspondence Address: LOEB & LOEB, LLP, 321 NORTH CLARK, SUITE 2300, CHICAGO, IL, 60654-4746, US

(73) Assignee: Huckleberry Toys, Northridge, CA (US)

(21) Appl. No.: 12/233,242

(22) Filed: Sep. 18, 2008

(51) Int. Cl. B65D 81/24; B32B 3/02; A61K 8/02; B65D 73/00; B65B 31/00; A61K 9/70; A01N 25/34

(52) U.S. Cl. 206/210; 442/59; 428/195.1; 442/123; 442/96; 442/164; 442/152; 442/153; 424/402; 424/404; 206/494; 53/511

(57) ABSTRACT

The invention relates to a pre-packaged, pre-moistened towel, which is customized. The towel is made from a woven cloth material, which can be customized by printing logos, designs or words on the towel. The towel can be either disposable or reusable.

The invention relates to a pre-packaged, pre-moistened, customized towel. The towel is made from a woven cloth material, which is customized by printing branded or corporate logos, characters, designs, events, words, savings or pictures on the towel. The towel can be either disposable or reusable.

### BACKGROUND OF THE INVENTION

Wet disposable towels are known in the art. Typically, they are made of fibrous paper and are stored in an airtight pouch such as an aluminum foil pouch lined with a protective liner such as polyethylene. When desired for use, a user tears open the pouch and removes the towel. Such towels are usually impregnated with an aqueous solution containing various additives such as perfumes, astringents, humectants and frequently menthol to give a cooling effect when applied to the skin. Towels of this sort are typically disposable and cannot be reused. Examples of such wipes include those disclosed in U.S. Pat. Nos. 3,786,615; 3,889,804; 6,103,644; 6,616,641 B2; 7,198,780 B2; and U.S. Patent Application Publication Nos. 2003/0029740 A1; 2003/0194932 A1; 2003/0199838 A1; 2006/0237474 A1; and 2007/0166402 A1.

### BRIEF SUMMARY OF THE INVENTION

The present invention relates to a pre-packaged, wet towel, wherein said towel is customized. The towel of the invention is made of a woven cloth. The customization of the towel can be via printing or incorporation of a design into the woven cloth. The customization can be the incorporation of branded or corporate logos, characters, designs, events, words, sayings or pictures on or in the towel. The towel can be permeated with purified water, benzalkonium chloride, isothiazoline or a derivative thereof, glycerin, perfume, and/or lotions. The towel can be either disposable or washable and reusable.

Other systems, methods, features, and advantages of the present invention will be or will become apparent to one with skill in the art upon examination of the following figures and detailed description. All such additional systems, methods, features, and advantages are included within this description, are within the scope of the invention, and are protected by the accompanying claims. Accordingly, the present invention is not restricted except in light of the attached claims and their equivalents.

### BRIEF DESCRIPTION OF THE FIGURES

FIG. 1 shows the packaging for the customized, woven, wet towel of the invention.

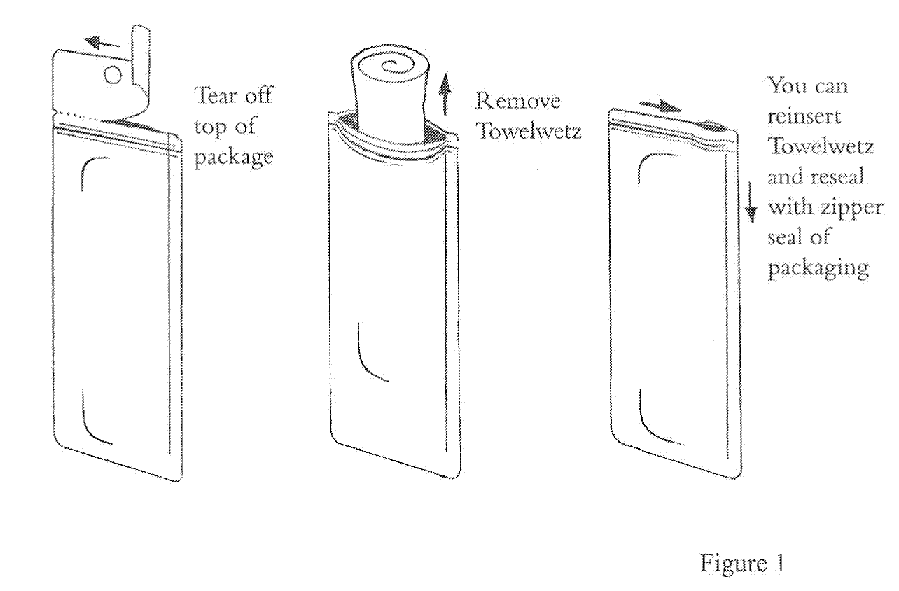

FIG. 2 is a diagram of the towel of the invention showing the placement of a corporate logo, artwork, design, character, saying or picture.

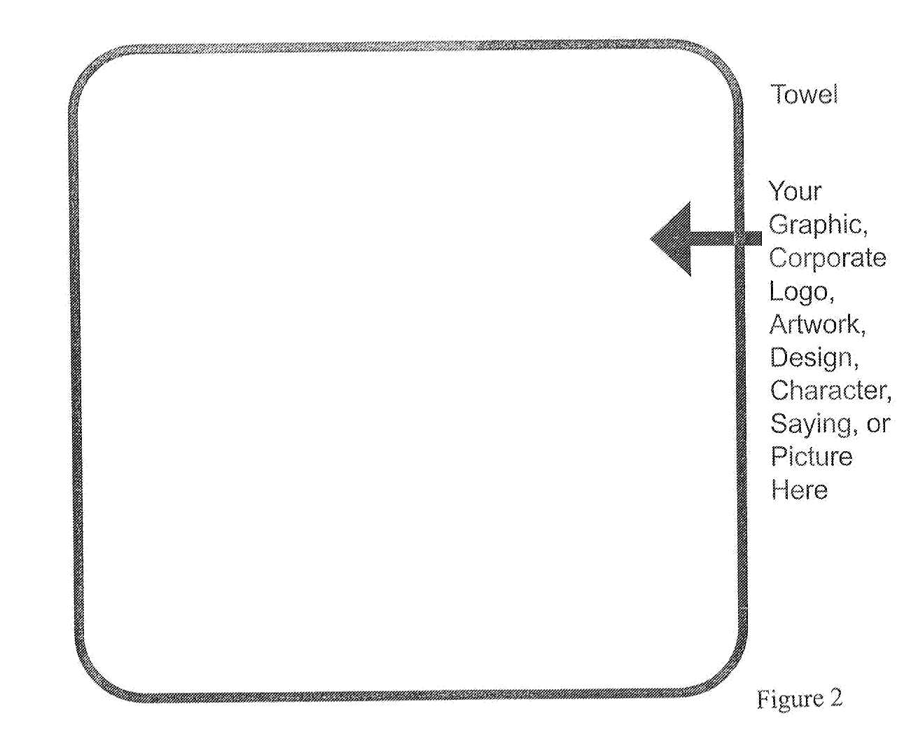

FIG. 3 is an example of a character towel.

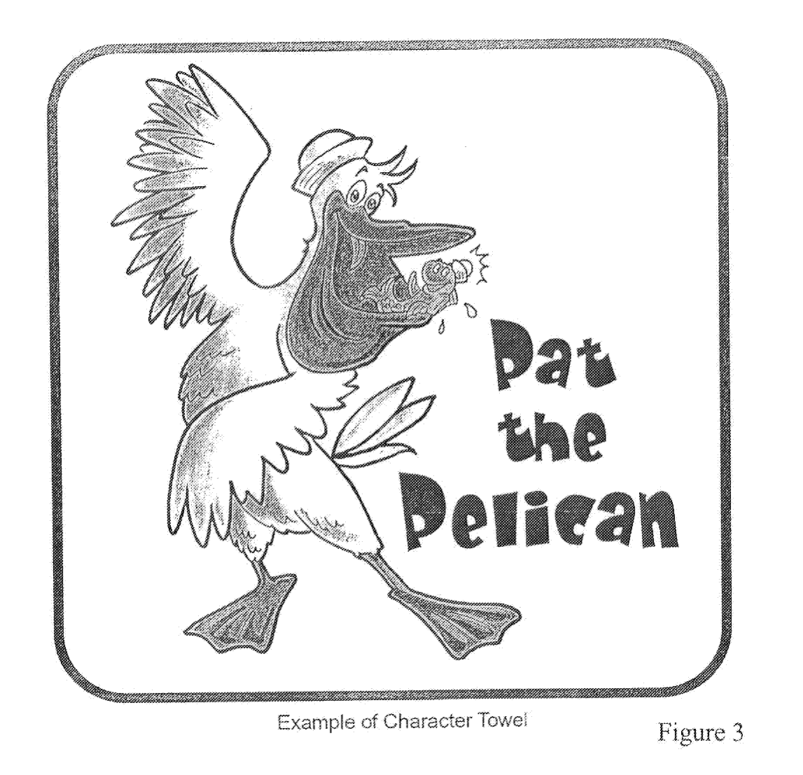

### DETAILED DESCRIPTION OF THE INVENTION

The invention relates to a customized, woven towel that can be pre-packaged as a wet towel. The woven material of the towel lends properties to a pre-packaged towel that are not found in ordinary pre-packaged paper wipes. Because the towel is made of a woven material, rather than plain paper or constructed from a web-like material, it is stronger and more durable. Further, the woven material can be used as a printing surface, or a design can be woven into the cloth. The towel of the invention is also washable and reusable, although it is inexpensive enough to be disposable or used over a limited period of time.

The term “woven” means a textile formed of interlaced threads, yarns, strips, fibrous material, or strands so as to form a fabric, cloth or material. The towel can be made of terry cloth, cotton, synthetic woven material, blends of different types of fibers, velour, micro-fiber, or wool. Typically, the towel is square in shape, although the towel could be made in other shapes, including, but not limited to, rectangles or circles.

The wet, woven towel of the invention is customized. The customization can be printed on the cloth, or woven into the cloth. The customization can be branded or corporate logos, characters, designs, events, words, sayings or pictures. Thus, the towel can be used for advertising a business, a function, an event, or as a personalized article.

The towel can be impregnated with purified water, benzalkonium chloride, isothiazoline or a derivative thereof, glycerin, perfumes, astringents, alcohols, humectants, menthol, antimicrobial compositions, disinfectants, lotions, emollients, medicaments, lubricants, skin protectants, tanning compositions, or cleansers.

The pre-packaged, customized wet towel can be used for a wide variety of purposes. For example, the towel can be used for personal cleansing or cooling; pet cleansing; cleaning or disinfecting objects, furniture or floors; or applying lotions or astringents.

The present invention can be prepared by any known method for manufacturing washcloths and towels. The pre-packaged wet towels would then be permeated with the desired liquid and packaged. The towels can be packaged individually or in multi-packs. If multi-packs are used, the towels can be removed for use one at a time. Alternatively, a multi-pack can hold a number of individually packaged towels. For single-towel removal from a multi-pack, the towels can be interfolded in a zig-zag pattern. Alternatively, some or all of the towels can be removed at one time from a multi-pack for use.

The wet towels of the invention can be vacuum-packed, which will increase the shelf-life of the towels. The vacuum-packaging can be an air-tight pouch, which can be lined with a protective liner. The package can be, but is not limited to, an aluminum-foil pouch. The protective liner can be, but is not limited to, polyethylene.

### EXAMPLE

The towel of the instant invention can be manufactured by any known means of preparing woven cloths. The customization can be incorporated into the cloth at the time of weaving or printed on the cloth after the manufacture is complete. Next, the cloth can be soaked in water or other desired liquid composition. After the cloth is moistened, it can be packaged via vacuum-packaging means. The vacuum-packaging can be an air-tight aluminum-foil pouch, which can be lined with a protective liner. The protective liner can be polyethylene.

While various embodiments of the present invention have been described above, it should be understood that such disclosures have been presented by way of example only, and are not limiting. Thus, the breadth and scope of the present invention should not be limited by any of the above-described exemplary embodiments, but should be defined only in accordance with the following claims and their equivalents.

Having now fully described the invention, it will be understood by those of ordinary skill in the art that the invention may be performed within a wide and equivalent range of conditions, formulations and other parameters without affecting the scope of the invention or any embodiment thereof. All patents, patent applications, and publications cited herein are fully incorporated by reference in their entirety.

I claim:

1. A pre-packaged, customized, wet towel, wherein said towel is made of a woven cloth.

2. The pre-packaged, customized, wet towel of claim 1, wherein the towel is customized by incorporation of branded or corporate logos, characters, designs, events, words, sayings or pictures.

2. The pre-packaged, customized, wet towel of claim 1, wherein the customization is printed on the towel.

3. The pre-packaged, customized, wet towel of claim 1, wherein the customization is woven into the cloth.

4. The pre-packaged, customized, wet towel of claim 1, wherein said towel is permeated with a liquid selected from the group consisting of purified water, benzalkonium chloride, isothiazoline, an isothiazoline derivative, glycerin, perfumes, astringents, antimicrobial compositions, disinfectants, self-tanning compositions, lotions, emollients, medicaments, lubricants, skin protectants and cleansers.

5. The pre-packaged, customized, wet towel of claim 4, wherein said liquid is water.

6. The pre-packaged, customized, wet towel of claim 4, wherein said liquid is glycerin.

7. The pre-packaged, customized, wet towel of claim 4, wherein said liquid is a perfume.

8. The pre-packaged, customized, wet towel of claim 4, wherein said liquid is benzalkonium chloride.

9. The pre-packaged, customized, wet towel of claim 4, wherein said liquid is isothiazoline or an isothiazoline derivative.

10. The pre-packaged, customized, wet towel of claim 1, wherein said towel is reusable.

11. The pre-packaged, customized, wet towel of claim 10, wherein said towel is washable.

12. The pre-packaged, customized, wet towel of claim 1, wherein said towel is disposable.

13. The pre-packaged, customized, wet towel of claim 1, wherein said towel is made of terry cloth, cotton, synthetic woven material, blends of different types of fibers, velour, microfiche, or wool.

14. The pre-packaged, customized, wet towel of claim 1, wherein said towel is vacuum-packaged.

15. The prepackaged, customized, wet towel of claim 1, wherein said towel is individually packaged.

16. The pre-packaged, customized, wet towel of claim 1, wherein said towel is packaged in a multi-pack.
